(19) United States
(12) Patent Application Publication
(10) Pub. No.: US 2024/0382443 A1
(43) Pub. Date: Nov. 21, 2024

(54) GENERATION OF S-NITROSOACETYLCYSTEINE

(71) Applicant: The UAB Research Foundation, Birmingham, AL (US)

(72) Inventors: Nagababu ENIKA, Birmingham, AL (US); Dan E. Berkowitz, Birmingham, AL (US)

(21) Appl. No.: 18/667,950

(22) Filed: May 17, 2024

Related U.S. Application Data
(60) Provisional application No. 63/502,714, filed on May 17, 2023.

(51) Int. Cl. A61K 31/198; A61K 9/00; A61K 33/00; A61P 9/12

(52) U.S. Cl. CPC A61K 31/198; A61K 9/0056; A61K 9/0065; A61K 9/0078; A61K 33/00; A61P 9/12

(57) ABSTRACT

The present disclosure is directed to compositions and methods for the generation of S-nitrosoacetylcysteine (SNOAC) for stable administration in the improvement, treatment and/or prevention of conditions that benefit from increased bioavailable nitric oxide. The present methods allow generation and delivery of SNOAC through various administration routes, including sublingually, orally, and through inhalation of aerosolized formulations. SNOAC is generated through a reaction between N-acetylcysteine and a nitrite compound in an aqueous medium.

### CROSS-REFERENCE TO RELATED APPLICATIONS

This application claims priority to U.S. Application No. 63/502,714, filed on May 17, 2023, which is incorporated herein by reference in its entirety.

### BACKGROUND

### Field of the Disclosure

The present disclosure relates generally to compositions and methods for rapidly generating S-nitrosoacetylcysteine (SNOAC), a nitric oxide (NO) donor. Related pharmaceutical compositions as well as methods of improvement, treatment, and prevention are provided.

### Background

Nitric oxide (NO) is an important signaling molecule for the function of the cardiovascular system. The NO synthesized by endothelial NO synthase regulates vascular tone and elicits several anti-atherogenic activities, such as decreasing platelet aggregation, reducing smooth muscle cell proliferation, and regulating vascular inflammation, by inhibiting adhesion of monocytes to endothelium. The bioavailability of NO begins to decline in humans at around the age of 30, and by around 70-80 years it is about two-thirds of its peak level. Decreased bioavailability of NO and the subsequent dysregulation of signal transduction pathways has been established as a major contributor to the development of cardiovascular disease. Hence, restoring NO levels in the vasculature and myocardium is an important potential therapeutic approach for preventing and/or treating cardiovascular disease.

The development of NO-donor drugs has been disappointing. The only approved NO-donor drug is sublingual nitroglycerin, which was developed a more than a century ago based on organic nitrates. Nitroglycerin is converted to NO by mitochondrial aldehyde dehydrogenase 2 (ALD2). The released NO dilates the blood vessels to retain more blood in the systemic circulation, thereby reducing stress on the heart and protecting against congestive heart failure. A significant problem with these drugs is that patients rapidly develop a tolerance, because the ALD2 enzyme becomes inactivated during bioactivation of the organic nitrates. In addition, chronic use of these drugs causes endothelial toxicity as toxic metabolites like cyanide and peroxynitrite accumulate.

Cysteine-NO, S-nitrosoglutathione (GSNO), and S-nitrosoproteins (albumin-SNO) are endogenous NO donors and potent vasodilators. These have longer half-lives than NO and transfer the bioactivity via guanylyl cyclase/cyclic guanosine monophosphate (cGMP)-dependent and-independent pathways. S-nitrosylation of proteins plays a key role in health and disease of the cardiovascular system. S-nitrosylated proteins release NO spontaneously without need for enzymatic bioactivation. Therefore, they do not produce tolerance or cause toxicity as occurs with organic nitrates. However, the pharmacologic activities of exogenous low molecular weight S-nitrosothiols (RSNOs), such as S-nitrosocysteine, S-nitroso-N-acetylcysteine (SNOAC), GSNO, and S-nitroso-N-acetylpenicillamine (SNAP) are underutilized because they are unstable after preparation. As such, a need remains for preparation of such RSNOs so that they may be delivered to patients before degradation.

### SUMMARY

The present disclosure describes an approach to on-demand generation of SNOAC that addresses the problems described above, although it is to be understood that not all embodiments described will address every such problem. The ability to generate SNOAC immediately prior to administration enables the practical use of SNOAC as a clinical agent before being degraded.

A first general embodiment is a pharmaceutical composition for the increasing levels of NO in a subject, the composition comprising inorganic nitrite and N-acetylcysteine (NAC) under moisture-free conditions. The inorganic nitrite can be, for example, sodium nitrite or potassium nitrite. In some embodiments, the composition further includes a chemical buffer to maintain the pH to circumneutral after formation of SNOAC. Some embodiments of the pharmaceutical composition find use in improvement, treatment, or prevention of a condition associated with low nitric oxide.

A second general embodiment is a composition for the rapid generation of SNOAC, the composition comprising nitrite and NAC under moisture-free conditions.

A third general embodiment is a method of improvement, treatment, or prevention of a condition that is benefited by increasing levels of NO, comprising administering SNOAC to a subject in need thereof. The SNOAC may be generated immediately prior to administration, during administration, or after administration by mixing nitrite and NAC in an aqueous environment, which may be a bodily fluid or may be extracorporeal.

A fourth general embodiment is a method of rapidly generating SNOAC, comprising: mixing nitrite and NAC in water. In some embodiments, the mixing occurs in a storage vessel. In some embodiments, the water is provided by a bodily fluid of a subject.

A fifth general embodiment is a method of improvement, treatment, or prevention of a condition that is benefited by increased nitric oxide, the method comprising co-administering NAC and nitrite to a subject in need thereof, and thereby producing SNOAC.

In some embodiments, the moisture-free conditions include storage in a sealed vessel. In some instances, the moisture-free conditions further include storage under nitrogen. In some embodiments, the inorganic nitrite and N-acetylcysteine are present at a nitrite: N-acetylcysteine ratio ranging from 1:20 to 1:10.

In some embodiments, the nitrite and N-acetylcysteine are present at a nitrite: N-acetylcysteine ratio ranging from 1:20 to 1:10. In some embodiments, the nitrite is present at a range of 2.5 to 10 mg and the N-acetylcysteine is 50 mg to 100 mg for an adult human subject, or subjects of comparable mass. In some embodiments nitrite is 2.5, 5, 10, 15 or 20 mg with corresponding 10 or 20 fold excess NAC.

The conditions that are benefited by increased NO include but are not limited to cardiovascular disease, pulmonary hypertension, pulmonary arterial hypertension, acute pulmonary hypertension-associated lung disease, chronic obstructive pulmonary disease, acute respiratory distress syndrome, lung infection, congestive heart failure, systemic hypertensive disease, vasoconstriction, poor circulation, erectile dysfunction, coronary artery disease, heart attack, stroke, atherosclerotic disease, vascular platelet adhesion, vascular platelet aggregation, monocyte adhesion to endothelium, leukocyte adhesion to endothelium, age-associated drop in endothelial function, age associated increase in arterial stiffness, peripheral artery disease, memory decline, decline in physical performance, and a combination of two or more of the foregoing. Furthermore, the methods and compositions here in may be used to improve a performance characteristic that is associated with NO, such as physical conditioning, cognitive function, memory, or a combination of two or more of the foregoing.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure.

FIG. 1 is a schematic depiction of an exemplary reaction vessel and an image of an exemplary nebulizer for the generation and delivery of SNOAC aerosols. In the reaction vessel, NAC and nitrite are present in a chamber without moisture and under nitrogen. In one example NAC is present in a dry solid form. After saline or water is added to the lower chamber, SNOAC is generated, which can be aerosolized using the nebulizer and administered in improvement, treatment, or prevention of conditions or diseases according to methods of the present disclosure.

FIG. 2 is a graphical representation of the anti-pulmonary hypertensive effect of inhaled aerosolized SNOAC in rats with severe Sugen-hypoxia-induced pulmonary hypertension. In the left panel, right ventricular systolic pressure (RVSP) is shown as a function of time for control animals (denoted with circular markers) and animals that were administered inhaled aerosolized SNOAC (denoted with squares). In the right panel, RVSP is shown as a function of time with a decrease sustained for approximately 5 minutes after cessation of the drug administration.

FIG. 3 is a schematic representation of typical (top) and present method (bottom) approaches for increasing NO bioactivity. The method of the present disclosure generates SNOAC, which decays to NO without needing enzymatic bioactivation. ALDH₂, aldehyde dehydrogenase 2; BP, blood pressure; NO, nitric oxide; NTG, nitroglycerin; SNOAC, S-nitroso-N-acetylcysteine.

FIG. 4 is a graphical representation of the transfer of the SNOAC that is generated in the sublingual space to blood circulation in rats. Either control (denoted with circular markers) or nitrite with NAC (denoted with square markers) was prepared and then administered to the sublingual space of rats. S-nitrosothiol concentrations were measured over time. Rats were sublingually administered 0.1 mg nitrite and 10 mg NAC in 50 μL of water, generating SNOAC.

FIG. 5 is a graphical representation of the transfer of SNOAC bioactivity from blood circulation to blood vessels, as assessed by femoral arterial blood pressure measurements. Downward pointing triangles represent rats treated with nitrite or NAC alone. In other groups, rats were sublingually administered SNOAC generated by reacting 50 μg (upward pointing triangles), 100 μg (circles), or 200 μg (squares) nitrite with a constant 10 mg NAC. Mean arterial pressure (MAP) change was measured over time for each group.

FIG. 6 are graphical representations of the generation and transfer of SNOAC to blood circulation following the oral administration of a mixture of sodium nitrite and NAC in neutral aqueous solution. In the left panel, control (squares), NAC only (circles), NO₂_{^{−}} (upward pointing triangles), and NAC with NO₂_{^{−}} (downward pointing triangles) represent the treatment groups of rats. Plasma S-nitrosothiol concentration was measured at 0.5,1, 2, or 3 hour time points after rats were sacrificed. In the right panel, S-nitrosothiol levels in rat livers were compared between control and NAC+nitrite groups.

FIG. 7 is a graphical representation of the decrease in systemic blood pressure after oral administration of a sodium nitrite−NAC mixture in neutral solution to normotensive rats. MAP was measured for rats treated with nitrite or NAC alone (downward pointing triangles), or a mixture of 10 mg NAC with 0.1 mg (upward pointing triangles), 0.2 mg (squares), or 0.4 mg (circles) nitrite.

FIG. 8 is a graphical representation of the antihypertensive activity of the combined mixture of sodium nitrite and NAC in the drinking water of eNOS^{-/-} transgenic hypertensive mice. MAP was measured for wild-type (WT), eNOS^{-/-} control (eNOS^{-/-} cont, and eNOS^{-/-}+nitrite+NAC mice (eNOS^{-/-} exp) after administration for 2 months.

FIG. 9 is a graphical representation of the antihypertensive activity of the combined mixture of sodium nitrite and NAC in the drinking water of mice with L-NAME—induced systemic hypertension. Mice were either control (no treatment with L-NAME), L-NAME fed alone (0.1 g/L), L-NAME (0.1 g/L) fed with NAC (0.5 g/L), L-NAME (0.1 g/L) fed with nitrite (20 mg/L), or L-NAME (0.1 g/L) fed with NAC (0.5 g/L) and either 10 mg/L or 20 mg/L of nitrite. MAP was measured after administration of doses for 2 months.

FIG. 10 is a graphical representation of the antihypertensive activity of the combined mixture of sodium nitrite and NAC in the drinking water of Spontaneously Hypertensive Rats (SHR). Rats were Normotensive Wistar Kyoto rats (WKY) as a control or Spontaneously Hypertensive Rats (SHR). Control rats were not fed with NAC or nitrite. Other experimental groups were administered nitrite, NAC, or a combination of NAC and nitrite in drinking water over 3 months, after which MAP was measured.

FIG. 11 is a graphical representation of generation and decomposition of S-nitroso-N-acetylcysteine (RSNO). In the left panel, RSNO generation is show when 15 mM sodium nitrite is reacted with 60 mM N-acetylcysteine (RSH) in water. All nitrite is converted instantly to RSNO. In the right panel, RSNO decomposition is monitored by recording spectra at 1.5 hour intervals over 15 hours. Approximately 50% of the RSNO was decomposed over the 15 hours.

FIG. 12 is a graphical representation of proteomics analysis of rat heart tissue. A single dose of nitrite and NAC at neutral pH were administered by oral gavage to rats. Rats were sacrificed after 1 hour and the hearts were collected. Total RSNO sites were identified using the a SNO-RAC (resin assisted capture) protocol. Proteomics was performed using liquid chromatography-tandem mass spectrometry (LC-MS/MS) analysis.

FIG. 13 is a graphical representation of the antihypertensive activity of the combined mixture of sodium nitrite and NAC in the drinking water of mice with L-NAME-induced systemic hypertension. Mice were either control (no treatment with L-NAME), L-NAME fed alone (0.1 g/L), L-NAME (0.1 g/L) fed with NAC (0.5 g/L), L-NAME (0.1 g/L) fed with nitrite (20 mg/L), or L-NAME (0.1 g/L) fed with NAC (0.5 g/L) and either 10 mg/L or 20 mg/L of nitrite. Arterial stiffness measured after administration of doses for 2 months.

FIG. 14 is a graphical representation of the transfer of the SNOAC that is generated in the sublingual space to blood circulation in mice. The concentration of total S-nitrosothiols at 1, 30 min., and 60 min. was determined after mice were sublingually administered 0.1 mg nitrite and 10 mg NAC in 50 μL of water, generating SNOAC.

FIG. 15 is a schematic representation of the results of sublingual administration of NAC and nitrite.

FIG. 16 is a schematic representation of an oral medication route where NAC pH is adjusted to 7.2 and mixed with nitrite, then administered by gavage to rats of the study.

FIG. 17 is a schematic representation of the results of oral administration of neutral NAC and nitrite.

FIG. 18 is a schematic representation of an experimental protocol for modelling dosage of NAC, nitrite, or NAC with nitrite for SHR rats or WKY rats. SNO: S-nitosothiols; MAP: Mean arterial pressure; PWV: pulse wave velocity.

FIG. 19 is a graphical representation of plasma S-nitrosothiols in WKY and SHR rats after dosage according to the experimental protocol of FIG. 18.

FIG. 20 is a graphical representation of arterial stiffness of rats treated with differing mixtures of sodium nitrite and/or NAC in the drinking water. Rats were WKY as a control or SHR. Control rats were not fed with NAC or nitrite. Other experimental groups were administered nitrite, NAC, or a combination of NAC and nitrite in drinking water over 3 months, after which arterial stiffness by measuring aortic pulse wave velocity (PWV) was measured. The Y axis shows arterial stiffness.

FIG. 21 are graphical representations of aortic vessel relaxation percentage as a function of acetylcholine (ACH). The treatment groups are NAC treatment (top panel), nitrite treatment (middle panel), and NAC with nitrite treatment (bottom panel).

FIG. 22 is a schematic representation of an experimental protocol for modelling the antihypertensive activity of the combined mixture of sodium nitrite and neutralized NAC in the drinking water of mice with L-NAME-induced systemic hypertension.

FIG. 23 is a schematic representation of an experimental protocol for sublingual administration of a sodium nitrate and NAC mixture to human subjects. Subjects are administered a low dose (2.5 mg sodium nitrite and 50 mg NAC) or high dose (5 mg sodium nitrite and 50 mg NAC) of the mixture calculated based on pre-clinical studies, where the dose decreases subject blood pressure modestly (less than 7%). If subject blood pressure decreases below safe levels, physicians take steps to increase it by administering a 0.5 L bolus of intravenous fluids with or without phenylephrine. Blood pressure is monitored throughout the two-hour study using a blood pressure cuff.

### DETAILED DESCRIPTION

### Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated.

With reference to the use of the word(s) “comprise,” “comprises,” and “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “including” should be interpreted to mean “including but not limited to . . . ” unless the context clearly indicates otherwise.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

The term “adapted to” means designed or configured to accomplish the specified objective, not simply able to be made to accomplish the specified objective.

The term “capable of” means able to be made to accomplish the specified objective.

The terminology used herein is for the purpose of describing particular embodiments only and is not intended to be limiting. As used herein, the singular forms “a”, “an” and “the” are intended to include the plural forms as well (i.e. “at least one”), unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The terms “prevention”, “prevent”, “preventing”, “suppression”, “suppress” and “suppressing” as used herein refer to a course of action (such as implanting a medical device) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as implanting a medical device) initiated after the onset of a clinical manifestation of a disease state or condition so as to eliminate or reduce such clinical manifestation of the disease state or condition. Such treating need not be absolute to be useful.

In this disclosure terms such as “administering” or “administration” include acts such as prescribing, dispensing, giving, or taking a substance such that what is prescribed, dispensed, given, or taken is actually contacts the patient's body externally or internally (or both). It is specifically contemplated that instructions or a prescription by a medical professional to a subject or patient to take or otherwise self-administer a substance is an act of administration.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or device of the present disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method or device of the disclosure.

Terms such as “at least one of A and B” should be understood to mean “only A, only B, or both A and B.” The same construction should be applied to longer list (e.g., “at least one of A, B, and C”).

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

None of the definitions above are intended to define what might be considered “equivalent” to anything that is claimed, under the “doctrine of equivalents” or analogous laws.

### Method of Generating Snoac

A method of rapidly generating SNOAC is disclosed. The reaction that generates SNOAC can be performed quickly and safely by persons with little or no training, allowing paramedical personnel and even patients to generate SNOAC as needed immediately prior to administration. The method involves mixing nitrite and NAC in water. In some instances, SNOAC is generated from a composition including nitrite and NAC when the composition is contacted with water, saliva, saline, or another aqueous solution. In some instances, the composition generates SNOAC when it is contacted with an acidic environment, such as gastric acid in the stomach of a subject.

Methods of rapidly generating SNOAC are provided with numerous advantages over existing methods. These advantages include increased SNOAC stability at administration, lower tolerance to the compounds, and lower toxicity to the subject of administration (although not all embodiments of the methods described herein will have all of these advantages, and the disclosure of these advantages is not intended to limit the invention to embodiments with any such advantages). In this context, nitrite includes inorganic nitrite (such as sodium nitrite), and other forms of nitrite (including other nitrite salts) capable of oxidation in the presence of NAC and water. Without wishing to be bound by any hypothetical model, it is believed that NAC releases hydrogen ions from its carboxyl group to serve as an acid to react nitrite with NAC to form SNOAC.

In some instances, NAC and nitrite are provided in a composition in equal amounts. In some instances, NAC and nitrite are provided in a composition at a nitrite: NAC ratio of 1:100, 1:50, 1:40, 1:30, 1:20, 1:10, 1:5 or 1:2 ratio.

The amount of nitrite and the amount of NAC may depend on the mass of a subject to whom the resulting SNOAC will be administered. For example, that amount of nitrite may be about 1-10 mg per 75 kg subject body. In specific embodiments of the method the amount of nitrite is any of 1 mg per 75 kg subject body, 2.5 mg per 75 kg subject body, 5 mg per 75 kg subject body, and 10 mg per 75 kg subject body. The amount of NAC may be about 10-100 mg per 75 kg subject body. In specific embodiments of the method the amount of NAC is any of 10 mg per 75 kg subject body, 25 mg per 75 kg subject body, 50 mg per 75 kg subject body, and 100 mg per 75 kg subject body. In a preferred embodiment, nitrite is used at no greater than 10 mg per 75 kg subject body. References to “nitrite” by mass refer to the molar equivalent of sodium nitrite unless stated otherwise.

The reaction may be conducted at a temperature suitable to accomplish the desired reaction rate and to provide the desired level of stability to the reaction products. Some embodiments of the methods proceed adequately at a temperature of about room temperature (approximately 20° C.) or higher. Some embodiments of the methods proceed adequately at approximately body temperature (approximately 37° C.). In some embodiments of the method the reaction is allowed to take place at a temperature >4° C. In further embodiments of the method the reaction is allowed to take place at a temperature ≥20° C. In further embodiments of the method the reaction is allowed to take place at a temperature up to 50° C.

Some embodiments of the method disclosed here produce no unsafe amounts of toxic NO₂, and therefore has the advantage of increased safety for human use in comparison to direct administration of NO, which must be undertaken in anaerobic conditions to avoid the formation of NO₂. Without wishing to be bound by any given hypothetical model, SNOAC releases NO at a very slow rate such that any NO₂ formed in the SNOAC reaction mixture or head space of a SNOAC reaction vessel converts back to nitrite as a result of fast reaction with water molecules. It is believed that in such embodiments NO₂ concentrations, if present at detectable amounts, are far less than that mandated by FDA safety limits. In some embodiments of the method, SNOAC produces no detectable NO₂. Some embodiments of the method result in a level of NO₂ that is safe for humans.

Other reaction conditions may be controlled to prevent degradation of the reaction products, maintain a sufficiently high reaction rate, or both. For example, in some embodiments of the method it is advantageous to use anhydrous conditions to increase stability of nitrite and NAC. Accordingly, some embodiments of the method include a composition stored under moisture-free or anhydrous conditions. In some such embodiments, moisture content is below 20, 19, 18, 17, 16, 15, 14, 13, 12, 11, 10, 9, 8, 7, 6, 5, 4, 3, 2, or 1 ppm w/w. In a further embodiment, composition storage conditions are anhydrous. In a specific embodiment of the method, the composition is stored under nitrogen or traces amount of oxygen until generation of SNOAC is desired.

In some embodiments of the method nitrite and NAC are allowed to passively mix. In some embodiments of the method the NAC and nitrite are actively mixed. Useful mixing methods include stirring, bead-beating, ball-milling, planetary milling, and co-extrusion.

### Compositions for the Generation of SNOAC

Reaction mixtures are provided that are useful for the methods described above. The reaction mixtures comprise NAC and nitrite. The forms of NAC and nitrite may be any that are described above as useful in the method. The concentrations of NAC and nitrite may likewise be any that are described above as useful in the method.

A composition containing a SNOAC-generating compound is provided that is the product of any of the methods described above. The compound includes nitrite and NAC, as described as being useful in the method above. The composition may also comprise metal chelators as suitable for use in the method.

As discussed above, the composition will in some embodiments generate NO₂ from NO at a level that is safe for human use. Some embodiments of the composition generates no more than 1000, 500, 250, 240, 230, 220, 210, 200, 190, 180, 170, 160, 150, 140, 130, 120, 110, 100, 90, 80, 70, 60, 50, 45, 40, 35, 30, 25, 20, 15, 14, 13, 12, 11, 10, 9, 8, 7, 6, 5, 4, 3, 2, and 1 ppb w/v NO₂ that is produced by SNOAC. A specific embodiment of the composition generates less than 100 ppb w/v of NO₂ that is produced by SNOAC during aerosolization in inspired oxygen. A further specific embodiment of the composition contains less than 15 ppb w/v of NO₂ that is produced by SNOAC. A further specific embodiment of the composition contains no detectable NO₂ that is produced by SNOAC, particularly when administered sublingually.

### Methods of Improvement, Treatment, and Prevention

The teachings of the present disclosure provide for the improvement, treatment and/or prevention of diseases or conditions that are benefited by increased NO. Such diseases and conditions include those that are not necessary the result of low NO, but may benefit from increased NO (for example, due to a beneficial reduction in blood pressure). Such diseases and condition also include that are characterized by low bioavailability of NO in a subject in need of such treatment. Examples of conditions and disease states that are benefitted by increased NO include without limitation: pulmonary hypertension, pulmonary arterial hypertension, acute pulmonary hypertension-associated lung disease, chronic obstructive pulmonary disease, acute respiratory distress syndrome, lung infection, congestive heart failure, systemic hypertensive disease, vasoconstriction, poor circulation, erectile dysfunction, coronary artery disease, heart attack, stroke, atherosclerotic disease, vascular platelet adhesion, vascular platelet aggregation, monocyte adhesion to endothelium, leukocyte adhesion to endothelium, age-associated drop in endothelial function, age associated increase in arterial stiffness, peripheral artery disease, memory decline, decline in physical performance, and a combination of two or more of the foregoing. Furthermore, the methods and compositions here in may be used to improve a performance characteristic that is associated with NO, such as physical conditioning, cognitive function, memory, or a combination of two or more of the foregoing. This disclosure also provides treatment of acute pulmonary hypertension, right heart failure and lung infections, such as antibiotic resistant infections, and infections pursuant to cystic fibrosis).

Some embodiments of the method comprise sublingual and/or oral administration of SNOAC to treat or prevent ischemic heart attack, congestive heart failure, hypertensive crisis, or a combination of two or more of the foregoing. Further embodiments of the method comprise slow-release sublingual and oral administration for the treatment or prevention of cardiovascular problems, peripheral arterial disease, or a combination of two or more of the foregoing. Further embodiments of the method comprise administering aerosolized SNOAC by inhalation to treat or prevent acute pulmonary hypertension, pulmonary infection, or a combination of both.

The method of improvement, treatment, and/or prevention comprises administering to the subject any of the active pharmaceutical ingredients (APIs) disclosed herein. The method will often further comprise identifying a subject in need of such improvement, treatment, or prevention.

Said administration is accomplished by direct administration of any of the APIs or by generating any of the APIs at or near the site of administration. Administration of SNOAC results in its dissolution into the circulatory system of a subject, where it decays to produce NO and increases the NO bioavailability of the subject. Administration of NAC and nitrite in the presence of water, saliva, or gastric acid generates SNOAC, which is delivered to the subject.

The method of improvement, treatment and/or prevention may include improvement, treatment and/or prevention of acute and chronic diseases and conditions. The form of administration may be adapted for acute or chronic dosage based on the acute or chronic nature of the disease or condition. For instance, aerosolized inhaled API or sublingual administration of any of the APIs disclosed herein can result in a rapid administration of SNOAC and corresponding increase in NO bioavailability. In other cases, oral delivery may be adapted for extended release of SNOAC, for example by using moisture barrier coatings and due to the dissolution effectiveness of the APIs in gastric acid.

### Pharmaceutical Compositions

Useful compositions of the present disclosure may comprise one or more APIs as described above. In one embodiment, such API are in the form of compositions, such as but not limited to, pharmaceutical compositions and medicaments. The compositions disclosed may comprise one or more of such APIs, in combination with a pharmaceutically acceptable carrier. Examples of such carriers and methods of formulation may be found in Remington: The Science and Practice of Pharmacy (20^{th} Ed., Lippincott, Williams & Wilkins, Daniel Limmer, editor). To form a pharmaceutically acceptable composition suitable for administration, such compositions will contain a therapeutically effective amount of a compound(s).

The pharmaceutical compositions of the disclosure may be used in the improvement, treatment and prevention methods of the present disclosure. Such compositions are administered to a subject in amounts sufficient to deliver a therapeutically effective amount of the API(s) so as to be effective in the improvement, treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, sublingual, subcutaneous, intravenous, topical, epicutaneous, oral, intraosseous, intramuscular, intranasal and pulmonary. The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the API and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by sublingual or intravenous administration, or locally such as by subcutaneous injection or by application of a paste or cream.

The compositions of the present disclosure may further comprise agents which improve the solubility, half-life, absorption, etc. of the compound(s). Furthermore, the compositions of the present disclosure may further comprise agents that attenuate undesirable side effects of the compounds(s). Examples of such agents are described in a variety of texts, such a, but not limited to, Remington: The Science and Practice of Pharmacy (20^{th} Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the APIs and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, and can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain antioxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The API may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly (ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the nucleic acid molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The API of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### EXAMPLES

### Example 1: Administration of Inhaled Aerosolized SNOAC for Treatment of Pulmonary Hypertension

Inhaled NO therapy is used for the treatment and management of life-threating pulmonary hypertension (PH). This therapy currently is available only in the intensive care units (ICUs) and operating rooms of major hospitals. However, immediate intervention is often needed for patients with severe respiratory problems (e.g., hypoxic lung failure, right heart failure, chronic obstructive pulmonary disease, acute respiratory distress syndrome) under emergency conditions. Here, a method is presented to synthesize and aerosolize SNOAC for inhalation using a nebulizer and deliver it directly to patients through a face mask. The inhaled aerosolized SNOAC is deposited in the alveolar spaces/conducting airways and then diffuses into blood vessels where it increases the bioavailability of NO. The method may prevent ICU admissions and avoid the need for expensive and invasive extracorporeal membrane oxygenation to save lives.

Sodium nitrite and NAC are stable individually but unstable when mixed together. However, the mixture is stable for a long time in the complete absence of moisture. Appropriate amounts of moisture-stripped sodium nitrite, NAC, and EDTA (a metal chelator) are stored in a septum-sealed glass vial under nitrogen. These chemicals are stable under these conditions for approximately 2 years. When NO is needed, SNOAC is generated by injecting an appropriate amount of saline into the vial.

This SNOAC solution is transferred to a nebulizer to generate aerosolized vapor for immediate inhalation. The anti-PH effect of this aerosolized SNOAC has been established in rat models of PH.

### Example 2: Generation and Delivery of SNOAC to Reduce Systemic Blood Pressure and Protect Against Congestive Heart Failure and Cardiovascular Disease

Sodium nitrite and NAC may be purchased from GMP commercial sources. The present methods generate SNOAC by adding water to a mixture of sodium nitrite and NAC at an equal ratio. The hydrogen ions released from the carboxyl group of NAC molecules are believed to serve as the acid to react nitrite with NAC to form SNOAC (Eqs. 1 to 4).

NAC-SH→NAC-S⁻+H⁺ (Eq. 1)

NO₂_{^{−}}+H⁺↔HNO₂+H⁺↔NO⁺-H₂O (Eq. 2)

NAC-S⁻+(NO⁺-H₂O)→NO+NAC-S′+H₂O (Eq. 3)

NAC-S′+NAC-S′→NAC-SS-NAC (Eq. 4)

Under specific conditions, the mixture of sodium nitrite and NAC is combined with an appropriate amount of disodium biphosphate or tri-sodium phosphate buffering chemical to generate SNOAC to automatically adjust the pH to around 6.0. With this process, the amount of SNOAC generated is 20% less than that generated in the absence of buffering agent.

Appropriate amounts of sodium nitrite and NAC are formulated into tablets under completely moisture-free conditions. Two types of tablets that dissolve in an aqueous environment are designed, one that dissolves quickly for rapid NO release to treat acute conditions and one that dissolves slowly for gradual NO release to treat chronic diseases. The tablet is protected from atmospheric moisture by coating the surface with an appropriate moisture barrier. The tablets are stable for at least 2 years.

The fast or slow dissolvable tablet is placed under the tongue for acute or chronic NO therapy, respectively. The tablet dissolves in the saliva and generates SNOAC, which diffuses from the mucosal membrane of the buccal cavity into the blood circulation and then to the blood vessels where it decays to NO without need for any enzymatic bioactivation. The NO then acts to dilate the blood vessels.

The same tablet can also be administered in an oral route. The gastric acid facilitates SNOAC formation in addition to the acidity contributed by NAC. The SNOAC generated in the gut region is absorbed into the blood circulation rapidly after first-pass hepatic elimination. Hence, the dose of the oral tablet is generally higher than the dose of the sublingual tablet.

Both orally and sublingually administered SNOAC has been shown to lower blood pressure in normotensive rodents and to have an antihypertensive effect in established hypertensive rodent models. Because tablets cannot be administered in animal studies, the powder form of nitrite and NAC mixture was dissolved in water to make SNOAC just before use in studies of sublingual administration. For oral administration by gavage, sodium nitrite was dissolved in neutralized NAC. Nitrite does not react with neutral NAC solution. However, the acidic conditions of the stomach contribute to reaction of nitrite with NAC to generate SNOAC.

### Example 3: Developing Aerosolized S-nitrosoacetylcysteine as an Nitric Oxide Donor for Acute Cardiopulmonary Complications Therapy

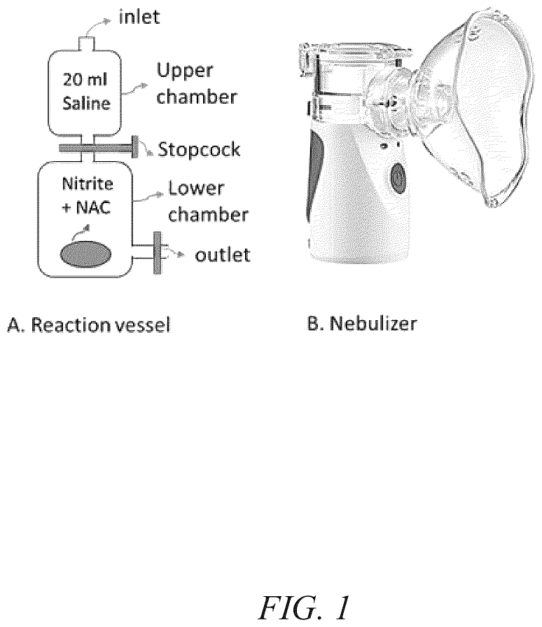

FIG. 1 illustrates a scheme for a disposable reaction vessel that can be used to generate SNOAC at need. It consists of two individual containers that are separated by an airtight rubber sealant stopcock. The upper container, with 25 mL volume capacity, has an inlet for introducing and storing saline. The 40-mL lower chamber stores the moisture-stripped SNOAC-generating chemicals. After the chemicals (NAC and nitrite) are placed in the lower chamber, the atmospheric air present is replaced with dry nitrogen gas from its outlet. These chemicals are stable in the anaerobic and completely moisture-free environment for at least approximately 2 years. When SNOAC is needed, the stopcock is turned to the “on” position, allowing liquid from the upper chamber to drop into the lower chamber, initiating the SNOAC-generating reaction within a few minutes. This SNOAC solution is transferred to a nebulizer through the outlet to convert into aerosol for inhalation. Alternatively, the lower chemical container is sealed with airtight septum. The chemical are stored under nitrogen. The liquid is injected into the container through the septum using needle syringe. The generated SNOAC is collected with the same syringe after a couple of minutes and then transferred into nebulizer. This procedure for generation and delivery of SNOAC to treat PH.

Stock SNOAC was prepared by reacting an appropriate amount of sodium nitrite and NAC in water just before use. The pH was adjusted to 7.0 with alkaline solution and then transferred to an Aeroneb Nebulizer (Kent Scientific). Severe PH was induced in Sprague Dawley rats by subcutaneous injection of vascular endothelial growth factor receptor antagonist Sugen 5416 followed by exposure to hypoxia (10% O₂) for 3 weeks and normoxia for 4 weeks. Right ventricular systolic pressure (RVSP) was measured invasively in a closed-chest, spontaneously breathing rat model as an indicator of pulmonary arterial pressure. Rats were anesthetized with 1.5% isoflurane in air with a flow rate of 2 L/min. A blood pressure catheter was inserted into the right ventricle through the jugular vein, and baseline RVSP was measured. After the pressure stabilized, the aerosolized drug was introduced into the isoflurane line for 5 minutes.

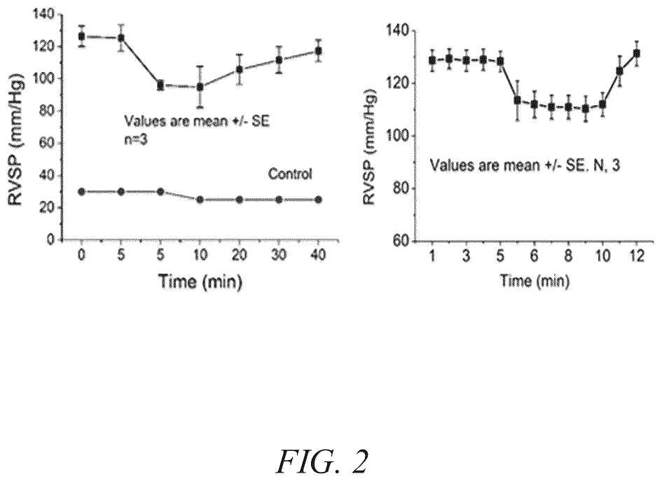

The baseline RVSP in control animals was 30 mmHg, as shown in FIG. 2, left panel. This RVSP did not change significantly after administration of aerosolized drug, indicating that this drug does not have any positive effect on normal blood pressure. The average baseline RVSP in hypertensive rats was 125 mmHg, approximately 4 times higher than that of control animals. Inhalation of aerosolized drug for 5 minutes resulted in an immediate decrease in RVSP from 125 to 96 mmHg. This decrease was sustained at the same level for 5 minutes after cessation of drug and then slowly increased over the following 40 minutes, while still remaining lower than the baseline value (FIG. 2). The subsequent administration of another dose of aerosolized drug after 40 minutes led to a sustained decrease in RVSP that was longer than after the initial dose. This RVSP decrease was compared with that produced by 30 ppm inhaled NO gas (FIG. 2, right panel). Inhalation of NO gas for 5 minutes resulted in a rapid decrease in RVSP. The RVSP remained constant during the period of inhalation but rapidly bounced back to baseline after cessation. Inhalation of precursor molecules of NAC or nitrite alone had no effect on RVSP. Sodium nitrite has been reported to reduce hypoxia-induced PH in animal studies. No effect was observed, even at higher doses in our study. Inhalation of SNOAC also significantly decreased angiotension-2—induced acute PH in control rats.

NO rapidly reacts with oxygen to generate NO₂, which is toxic to lungs. The FDA safety limit for the co-delivery of this gas with inhaled NO is <1 ppm. NO that is complexed with thiol groups does not react with oxygen. However, NO that is dissociated from SNOAC reacts with oxygen to generate NO₂. The present system is designed so that any NO₂ formed in the reaction mixture is immediately converted back to nitrite following the reaction with water. Therefore, the presence of NO₂ in the reaction vessel headspace and in SNOAC aerosols was measured. Measurements showed that no NO₂ was present in the reaction vessel headspace. NO does dissociate from SNOAC, albeit at a slow rate. This NO can react with oxygen to generate NO₂. The pharmacologic dose of 100 mM SNOAC was prepared and transferred to a nebulizer. The vapor that is produced at the rate 0.2 mL/min by the nebulizer is flushed out with nitrogen or air or 100% oxygen (4 L/min). Any NO released from SNOAC during this process can react with oxygen to generate NO₂. Therefore, NO and NO₂ in the aerosols was measured using a NO analyzer (chemiluminescence method), and CAPS NO₂ analyzer respectively. The SNOAC aerosols that exited the nebulizer were passed through a Drierite moisture trap cartridge (United Filtration Systems) to retain all the SNOAC vapor and passing NO and NO₂ gases before entering into the analyzers to measure the NO and NO₂ simultaneously. The results are shown in Table 1. There was about 5.5 ppm NO in carrier gas, indicating that this amount is released from the SNOAC. This NO is converted to NO₂ in the range of 12 ppb, 26 ppb, and 86 ppb in nitrogen, air, and 100% oxygen carrier gases, respectively. This amount of NO₂ is far less than the 1 ppm safety limit enforced by the FDA. Therefore, formation of toxic NO₂ during inhalation therapy of aerosolized SNOAC is not a concern.

TABLE 1
Nitric oxide and nitrogen dioxide levels
in the S-nitrosoacetylcysteine aerosols

| | | |
|---|---|---|
| Carrier gas | Nitric oxide (NO) | Nitrogen dioxide (NO₂) |

| | | | | |
|---|---|---|---|---|
| Nitrogen | 5.5 | ppm | 12 | ppb |
| Air | 5.6 ± 1.2 | ppm | 26 ± 5 | ppb |
| Oxygen | 5.2 ± 0.9 | ppm | 86 ± 8 | ppb |

These results suggest that inhalation of aerosolized RSNO by rats with Sugen-hypoxia-induced PH decreases RVSP by decreasing pulmonary arterial resistance. This effect would improve blood flow from the right ventricle to the pulmonary blood vessels. Aerosolized SNOAC can be deposited in the alveolar spaces/conducting airways and then rapidly diffused into lung tissue to increase NO bioavailability. These results indicate that aerosolized RSNO can treat acute and chronic pulmonary hypertensive diseases. Using SNOAC as an anti-pulmonary hypertensive agent has several advantages over inhaled NO. Inhaled NO rapidly reacts with oxygen to generate toxic NO₂. Therefore, it is advantageously generated under strict anaerobic conditions and diluted approximately 1250 times in nitrogen gas, compressed for storage in tanks, and supplied to hospitals. In contrast, SNOAC can be prepared in some cases within a few minutes wherever and whenever it is needed. SNOAC releases NO at a very slow rate. Any NO₂ formed in the SNOAC reaction mixture or head space of the reaction vessel converts back to nitrite as a result of high solubility and fast reaction with water molecules. The NO₂ concentration formed in the carrier gas is far less than that mandated by FDA safety limits. (2) With the inhaled NO delivery system, a specialized delivery and monitoring system is needed to introduce the diluted NO into the supplemental oxygen in a way that minimize NO₂ formation. In the SNOAC system, a fixed concentration of SNOAC is generated and the amount of aerosolization for dosing can be adjusted at the nebulizer. Another drawback of inhaled NO is that the PH rebounds back to baseline level rapidly after the withdrawal of treatment. With SNOAC treatment, the treatment effect is expected to be more sustained. Inhaled NO treatment is available only at major medical centers and is very expensive, whereas some embodiments of a SNOAC delivery system can be made available wherever it is needed at lower expense.

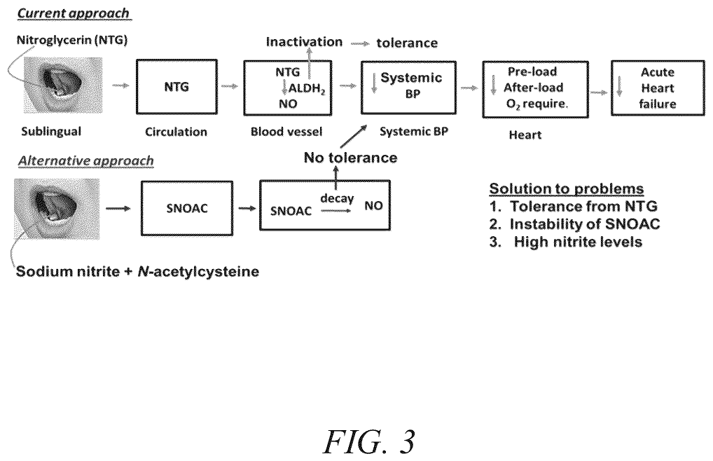

Sublingual administration was investigated. Sublingually administered organic nitrates of previous studies diffuse into the blood circulation from the buccal cavity, bypassing hepatic clearance, and then diffuse into the blood vessels where they are converted to NO by ALD2 enzyme. This NO dilates the blood vessels to retain more blood in the systemic circulation, thereby reducing the cardiac preload and afterload. NO also improves the coronary artery blood flow, thereby reducing cardiac stress and protecting against angina and congestive heart failure (FIG. 3). During the process of drug bioactivation, the enzyme gets deactivated, producing tolerance within 12 to 24 hours. This tolerance is the major hindrance to use of organic nitrates as a source of NO for continuous therapy.

In contrast to organic nitrates, it is believed that SNOAC of the present disclosure decays spontaneously to NO without need for enzymatic bioactivation (FIG. 3). Hence it would not produce tolerance with continuous treatment. The major drawback with SNOAC is that it is not stable after preparation, so there are currently no viable oral preparations for clinical usage. To address this problem, a system was developed whereby SNOAC is generated in the buccal cavity itself by reacting nitrite with NAC. The SNOAC is used immediately without need for storage. This procedure addresses the problem of SNOAC instability after preparation (FIG. 3).

This procedure also addresses the problem of requiring high doses of inorganic nitrite for use as an NO donor. Several in vitro and preclinical studies have shown that inorganic nitrite is converted to NO by cellular hemeproteins; thus, it has been advocated as an NO donor. Subsequent human clinical trials indicated that hemeproteins are not efficient converters of nitrite to NO. Therefore, high doses of nitrite would be needed to improve the vascular tone. The present technique converts 70% to 90% of nitrite to NO in the form of SNOAC in the buccal cavity; therefore, fewer milligrams of inorganic nitrite are needed.

Approaches to using SNOAC instead of organic nitrates may address the problems of organic nitrate tolerance, instability of RSNO after preparation, and the requirement of high concentrations of inorganic nitrites. Feasibility of SNOAC use was investigated in rodent animal models. The administration of sodium nitrite and NAC chemicals in powder or tablet form is not practicable in animal studies. Therefore, the procedures for mixing of nitrite with NAC for administration were modified for animal studies. For sublingual administration, the desired amount of nitrite and NAC were mixed in water (as a substitute for saliva) just before use and then administered sublingually to study the vasodilatory effect. For oral administration, the pH of the aqueous NAC solution was adjusted to 7.0 before it was mixed with sodium nitrite. Nitrite does not react with NAC in neutral conditions. This solution was administered by gavage for acute dosing studies or mixed in drinking water for chronic dosing studies.

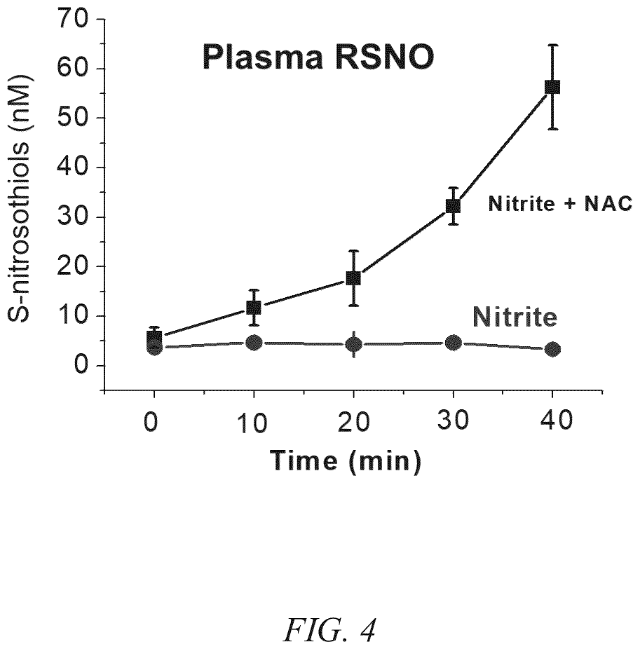

Because the chemicals cannot be placed in the buccal cavity of experimental animals, SNOAC was prepared in a test tube just before use and then placed in the sublingual space. Normotensive rats were anesthetized and the femoral artery was cannulated to collect blood. Stock SNOAC at 30 μmoles/mL was prepared by reacting sodium nitrite with NAC at the molar ratio of 1:2 in water just before use. It is believed that all of the nitrite is converted to SNOAC under these conditions. After baseline blood samples were collected in heparinized tubes, approximately 50 μL of SNOAC solution (0.4 mg nitrite and 1.87 mg NAC/kg body weight) was placed under the tongue of anesthetized rats. Blood was collected at 10-minute intervals. The plasma was separated, and nitrite and total S-nitrosothiols in plasma were determined. As shown in FIG. 4, total RSNOs in plasma increased over time from 6.5 nM at baseline to 65 nM at 40 minutes. These results suggest that the SNOAC diffuses from the buccal cavity to systemic circulation, where part of it can undergo transnitrosylation reactions with other sulfur groups of plasma proteins to form S-nitrosylated proteins and cysteine amino acid to form S-nitrosocysteine. There was no change in plasma nitrite levels. This experiment demonstrates the transfer of SNOAC from the buccal cavity to the bloodstream.

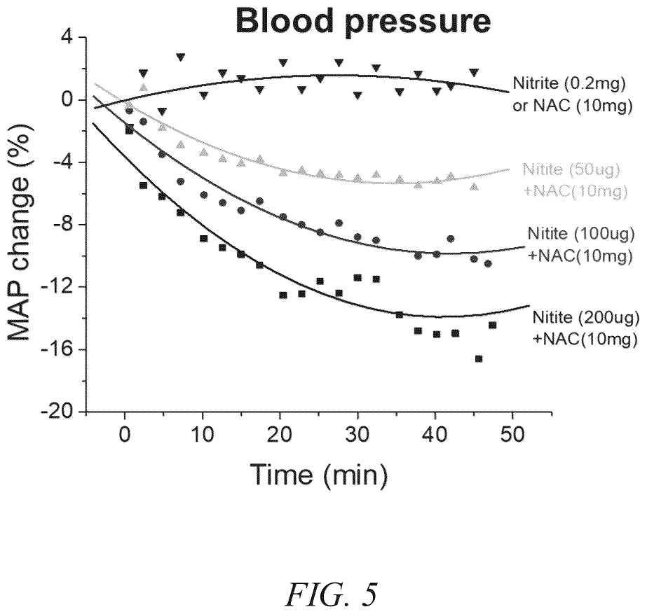

Normotensive rats were anesthetized with isoflurane. The femoral artery was catheterized with a Micro-Tip Catheter Transducer (AD Instruments, Houston, TX) for monitoring of blood pressure. Once the baseline blood pressure was stabilized, at approximately 30 minutes, rats were treated sublingually with 50 μL of SNOAC generated by reacting 0.2 mg, 0.4 mg, or 0.8 mg nitrite/kg body weight with a fixed concentration of 10 mg NAC (FIG. 5). Control rats were treated with nitrite or NAC alone. The mean arterial pressure (MAP) was continually monitored. There was no change in the MAP after administration of NAC or nitrite alone. However, the MAP decreased dose-dependently when rats were administered a combination of nitrite and NAC. The results indicate that SNOAC was absorbed from the buccal cavity, increasing plasma RSNO, and that the NO bioactivity of the RSNO dilated blood vessels to reduce blood pressure. It is believed that SNOAC and cysteine NO can diffuse into blood vessels and dilate the blood vessels via the classic cGMP-mediated or cGMP-independent post-translational modification of some of the key proteins of the signaling mechanism.

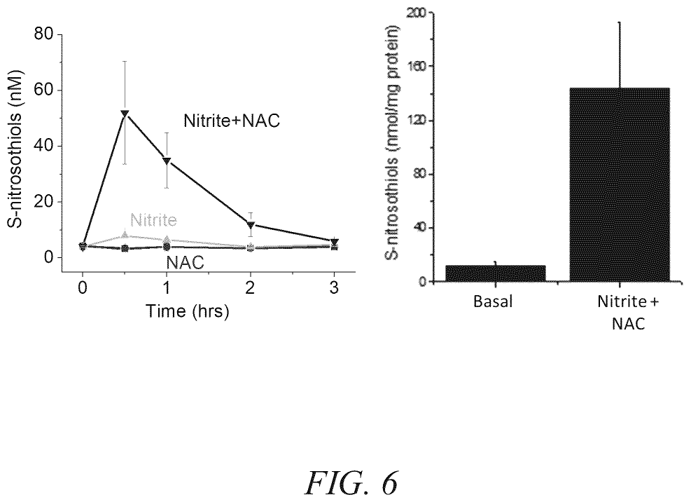

NAC is acidic in aqueous solution. Hence, the pH of NAC was adjusted to 7.0 with sodium hydroxide to prevent immediate reaction with nitrite when mixed. The acidic conditions of the stomach may be suitable conditions to react nitrite with NAC to generate SNOAC. Nitrite (1.5 mg/kg body weight) was dissolved in neutral NAC (50 mg/kg body weight) and administered to rats by gavage. The rats were sacrificed at 0.5, 1, 2, or 3 hours after drug administration. Then blood, liver, heart, and kidneys were collected. The plasma was separated for measurement of the RSNOs, as shown in FIG. 6, left panel. Organs were homogenized in a buffer containing NEM and EDTA, and RSNOs were determined on the same day without storage. There was a 2-fold increase in plasma RSNOs in the group treated with nitrite alone. There was no change in the group treated with NAC alone. However, in animals that were administered the solution of nitrite and NAC together, RSNOs increased to 8.5-fold over baseline within a half hour and slowly declined back to baseline levels within 3 hours. These results indicate that the stomach acid was sufficient for nitrite to react with NAC to generate SNOAC. This SNOAC was absorbed rapidly into the circulation and metabolized within 3 hours. There was no significant change in RSNO levels of the heart and kidneys. However, RSNO levels in the liver were increased to 14-fold over the control group (FIG. 6, right panel). These results suggest that the major portion of the SNOAC generated in the stomach is absorbed primarily to the liver through a portal vein and undergoes significant first-pass hepatic clearance before entering into the systemic circulation.

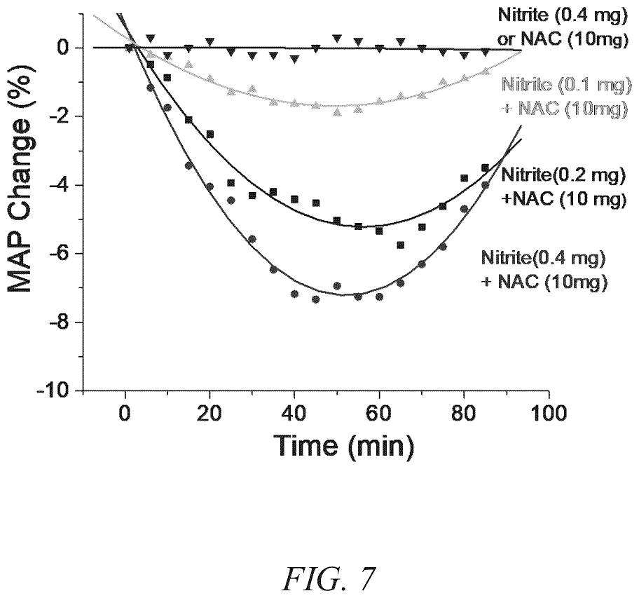

The MAP in rats was measured as described for sublingual administration. After baseline blood pressure was measured, 0.5 mL of nitrite alone, NAC alone, or the nitrite−NAC mixture was administered by gavage. The MAP decreased rapidly after administration of the nitrite−NAC mixture in a manner dependent on the nitrite dose (FIG. 7). It reached a nadir within 45 minutes and recovered to baseline level by 100 minutes. The dose required to reduce MAP was higher than the dose needed with sublingual administration. These results suggest that the RSNO in plasma after oral administration of nitrite along with NAC transfers its bioactivity to vasculature to reduce the systemic blood pressure. Administration of neither nitrite nor NAC alone altered the blood pressure. However, administration of nitrite in combination with NAC increased the efficiency and potency of nitrite. The stomach acid may play a role in formation of SNOAC under these conditions. Similar results are expected with oral administration of a tablet form of sodium nitrite and NAC.

### Example 4: Developing SNOAC as an NO Donor for Chronic Therapy to Protect Against Systemic Hypertension

The chronic dosing effect of sodium nitrite, NAC, or the combination of these two chemicals on systemic hypertension was investigated in a rodent model of hypertension by administering them in drinking water. Chronic administration of sodium nitrite together with NAC form SNOAC in the acidic gastric region, thereby enhancing tissue NO levels, improving vascular function, and reducing the incidence of cardiovascular disease. The antihypertensive effect of these chemicals was investigated in three hypertensive rodent models.

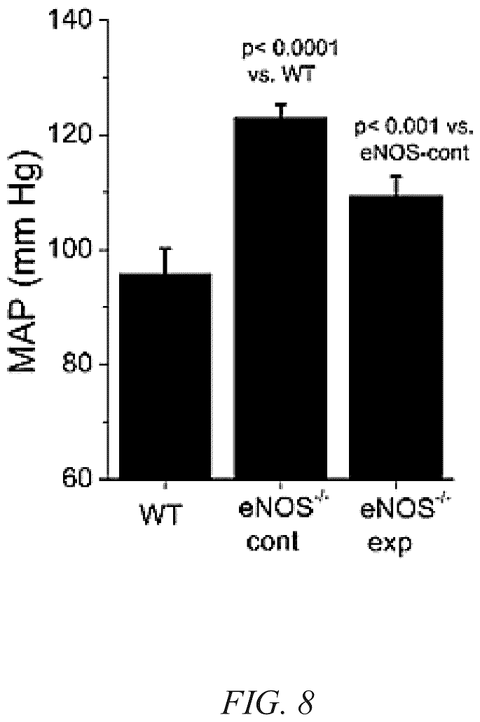

eNOS^{-/-} mice are hypertensive because they lack NO synthesis in blood vessels. 15-week-old wild-type (WT) and eNOS^{-/-} mice were divided into 5 groups. (1) WT control, (2) eNOS^{-/-} control, (3) eNOS^{-/-}+nitrite, (4) eNOS^{-/-}+NAC, and (5) eNOS^{-/-}+nitrite+NAC. NAC was dissolved in the drinking water, and pH was adjusted to 7.0 to prevent initial nitrite reaction with NAC. The mice were given pharmacological doses of nitrite (50 mg/L) and NAC (500 mg/L) individually or in combination for 2 months. Mean arterial pressure, which was measured by the tail-cuff method, was significantly higher in eNOS^{-/-} control mice than in WT mice (FIG. 8). Mice treated with nitrite or NAC alone exhibited no significant change in MAP. However, eNOS^{-/-} mice fed nitrite in combination with NAC had significantly reduced MAP compared with the eNOS^{-/-} control mice (FIG. 8). Thus, oral delivery of nitrite in combination with NAC represents a novel approach to restore the bioavailability of NO in vivo as reflected in improved MAP in eNOS^{-/-} mice.

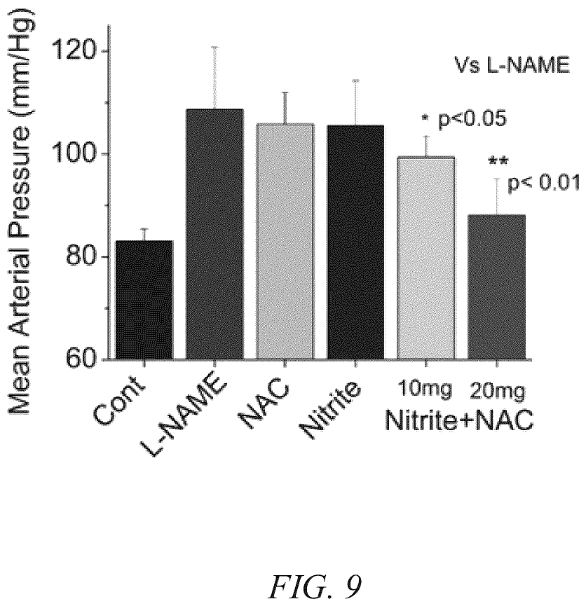

L-NAME (N-nitro-L-arginine methyl ester) is an inhibitor of eNOS enzyme activity. Administering L-NAME in the drinking water of animals induces systemic hypertension by decreasing NO synthesis in blood vessels. In this study, mice were fed L-NAME alone (0.1 g/L) or L-NAME+NAC (0.5 g/L), L-NAME+nitrite (20 mg/L), or L-NAME+NAC with one of two nitrite doses (i.e., 10 mg/L or 20 mg/L) in drinking water for 2 months. Mean arterial blood pressure was measured by the tail-cuff method (Kent Scientific, Torrington, CT). As shown in FIG. 9, the MAP of mice fed with L-NAME increased from the baseline of 82 mmHg to 110 mmHg. L-NAME-treated mice administered NAC or nitrite alone exhibited no significant change in MAP. However, MAP was significantly decreased in L-NAME—treated mouse groups administered 10 mg/L and 20 mg/L nitrite in combination with NAC. NAC increased the efficacy and potency of nitrite via generation of SNOAC in the stomach, and this SNOAC was then absorbed into the circulatory system. These results show that chronic oral administration of dietary components of inorganic nitrite (food additive) combined with NAC (antioxidant) is an alternative source of NO to protect against NO deficiency—associated cardiovascular diseases.

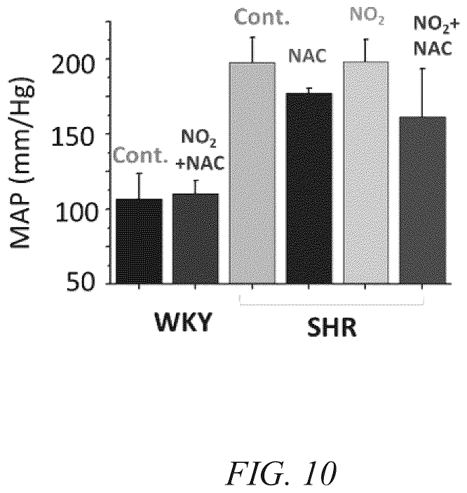

SHR rats are a well-established model used to study essential hypertension. Normotensive Wistar Kyoto rats (WKY) were used as a control group. Rats were fed with NAC (1.2 g/L), nitrite (60 mg/L), or a combination of the two in drinking water for 3 months. Mean arterial blood pressure was measured by the tail-cuff method once each month. At the end of 3 months (FIG. 10), MAP in the SHR control group was 1.8-fold higher than that in the WKY control group. MAP was modestly decreased in the NAC-treated SHR group, and nitrite treatment had no effect. However, MAP in the SHR group fed the nitrite−NAC combination was significantly (p<0.02) decreased compared with that of the SHR control group. The WKY mice fed the nitrite−NAC combination exhibited no change in MAP. These results suggest that the antihypertensive effect of nitrite+NAC is more effective in the hypertensive models of eNOS^{-/-} mice and L-NAME—treated mice than in the SHR model. The exact mechanism for inducing systemic hypertension in the SHR model is not known, but it appears that NO donors are less effective.

Administration of acute doses of sodium nitrite in combination with NAC sublingually or orally to normotensive rats resulted in rapid reduction of systemic blood pressure via generation of the NO-donor compound SNOAC. The sublingual route is more effective than the oral route because SNOAC diffuses directly into the bloodstream, bypassing the hepatic first-pass elimination. The efficacy and the potency of this drug make SNOAC an attractive drug choice to treat congestive heart failure.

Chronic dosing with a combination of nitrite and NAC in the drinking water of hypertensive rodent models led to a dose-dependent decrease in systemic blood pressure. This antihypertensive activity of SNOAC was more effective in the eNOS^{-/-} and L-NAME—treated mouse models of hypertension, which are induced by a decrease in NO synthesis. These results suggest that chronic oral administration of a SNOAC-generating system is a valuable strategy to be considered to increase endogenous NO levels. Apparently this approach does not result in the development of tolerance. As a result, this approach could compensate for the decrease in endogenous NO levels that occurs with aging and in several cardiovascular diseases.

It was observed that moisture-stripped crystals of the sodium nitrite and NAC mixture stored in a completely moisture-free environment are highly stable for at least 2 years, as they generate the expected level of SNOAC upon dissolution in water.

In conclusion, the method of SNOAC generation in the buccal cavity and in the stomach to increase endogenous NO addresses the problems posed by tolerance development, instability of SNOAC after preparation, and high concentrations of sodium nitrite required when used alone as an NO donor.

### Example 5: S-nitrosothiols as Nitric Oxide Donors to Protect against Cardiovascular Disease Risk Factors of Endothelial Dysfunction, Arterial Stiffness and Hypertension

Acute doses of nitrite (0.1 to 1 mg/kg B. wt.) or NAC (10 to 50 mg/kg B. wt.) alone or in combination were administered sublingually or by oral gavage to anesthetized Wistar rats and blood collected at varying time points for measurement of total plasma SNOs by chemiluminescence method. BP was measured by femoral intra-arterial catheterization. Chronic studies were carried out in an L-NAME (eNOS inhibitor) induced hypertensive mouse model. Mice were randomized into five groups 1) control, 2) control L-NAME (0.1 g/L), 3) L-NAME+nitrite (20 mg/L), 4) L-NAME+nitrite (10 mg/L)+NAC (0.5 g/L), 5) L-NAME+nitrite (20 mg/L)+NAC 0.5 g/L) in drinking water for 2 months. Plasma S-nitrosothiols blood pressure (tail cuff method), arterial stiffness (Doppler method) were determined.

In sublingual doses, total plasma SNOs levels increased with time and concomitantly blood pressure decreased in a dose-dependent manner suggesting that SNOAC generated in sublingually diffuses through mucosal membrane into the circulation and decreases the BP. In oral doses, plasma SNOs levels increased 10 fold over 30 min in nitrite−NAC treated rats and then returned to the baseline level by 3 h following administration. The systemic BP decreased in a dose-depended manner and returned to baseline level by 2 h. In chronic studies, treatment of mice with the combination of nitrite with NAC significantly decreased L-NAME induced hypertension and arterial stiffness, but not in mice treated with either nitrite or NAC alone.

Membrane diffusible S-nitroso-N-acetylcysteine (RSNO—synonymous with SNOAC) was chosen as a NO donor. This RSNO is prepared by reacting sodium nitrite (NaNO₂_{^{−}}) with N-acetylcysteine (RSH—synonymous with NAC) in water according to Eqs. 5 and 6:

RSH+NO₂_{^{−}}→RSNO+H₂O (Eq. 5)

RSNO→RSSR+NO (Eq. 6)

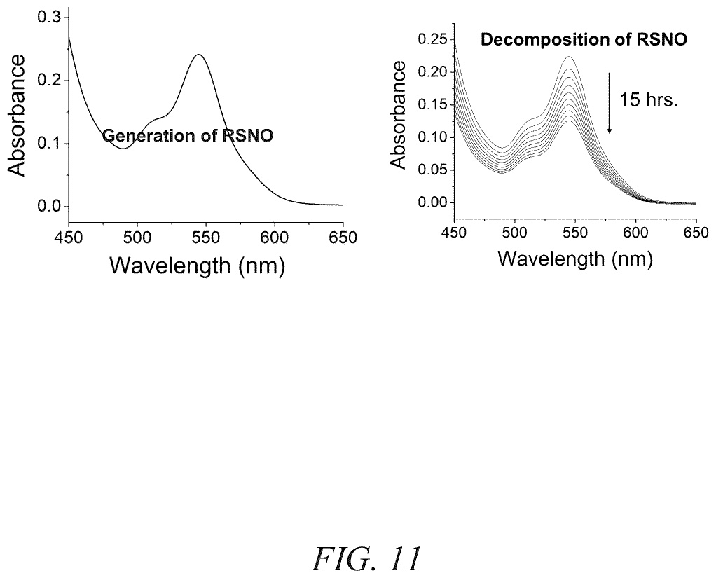

Nitrite is converted to nitric oxide gas by reacting with RSH in water. RSNOs have potent vasodilatory, anti-inflammatory, and antiplatelet activities. As shown by the comparison of generation of RSNO in FIG. 11, left panel, with degradation in FIG. 11, right panel, the instability of RSNO after preparation is a major hindrance to use them as NO donors to restore the NO bio-availability that decrease with aging and pathophysiological conditions of cardiovascular diseases. Therefore, RSNOs have proven easy to prepare, but not typically stable after preparation.

RSNO administered sublingually diffuses rapidly through the mucosal membrane to systemic circulation and reduces systemic systolic and diastolic blood pressure, as shown in FIG. 4 and FIG. 5.

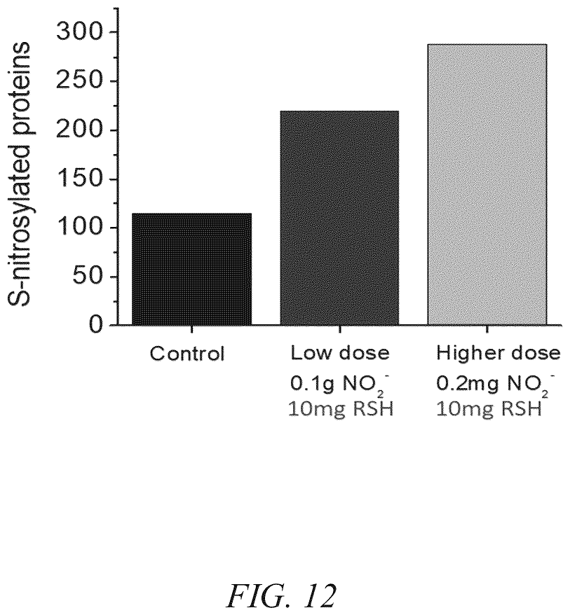

Oral administration of nitrite and RSH was then undertaken with rats, where the mixture was at a neutral pH. Co-administration of nitrite and RSH orally increased plasma RSNO and concomitantly decreased the mean arterial blood pressure, as shown in FIG. 6 and FIG. 7, respectively. The acidic conditions of the stomach provides ideal conditions for the reaction of nitrite with RSH to generate RSNO. This RSNO absorbs into the circulation and tissues where it releases NO (FIGS. 6-7). In an analysis of proteomics with rat heart tissue, S-nitrosylation of cardiac proteins increased with increasing the dose of RSNO, suggesting that RSNO diffuses from the circulation to blood vessels and then to myocardium (FIG. 12).

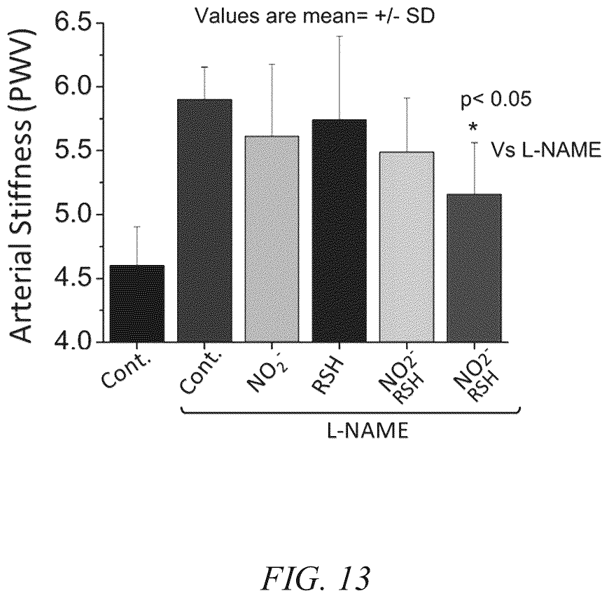

In chronic studies, oral doses of nitrite and RSH in combination decreases blood pressure and vascular stiffness more than either administered individually. (FIG. 9 and FIG. 13, respectively).

Herein, a procedure has been developed to generate and deliver S-nitrosothiols as a source of exogenous nitric oxide that can transfer the nitric oxide bioactivity to blood vessels and myocardium. Acute doses of sublingual and oral doses of nitrite and NAC in combination increase plasma SNOs levels and concomitantly decrease BP more than either administered individually. Delivery of nitrite and NAC through drinking water also increased plasma SNOs and decreased BP and arterial stiffness. Nitrite reacts with NAC under the acidic conditions of sublingual and in stomach to generate SNOAC, which is absorbed rapidly into the systemic circulation, raises plasma SNOs levels, and decreases the BP. SNOAC is an alternative to nitroglycerin-based drugs as a source of NO without developing tolerance and toxicity.

### Example 6: S-nitrosothiols-Based Therapeutic Approaches for the Management and Treatment of Cardiovascular Diseases

Aging is associated with a decrease in endothelial function and increases in arterial stiffness and blood pressure. These are major risk factors for development of cardiovascular disease (CVD), including atherosclerosis, coronary heart disease, arterial thrombotic disorders, and heart failure. While the exact etiology of these factors is not known, multiple factors play a role in development of these disorders. Several studies have shown that a decrease in NO bioavailability is one of the major factors contributing to the development of these risk factors.

Restoration of this NO bioavailability is considered to protect against CVD. Endothelial dysfunction, which characterizes vascular aging, is strongly associated with lower NO bioavailability, resulting in impaired vasodilatation, increased plaque formation and thrombosis. Several mechanisms are involved in reduced NO availability in aging. First, endothelial nitric oxide synthase (eNOS) activity, and therefore eNOS-derived NO production, decline with increasing age; second, excess reactive oxygen species (ROS) produced by arteries during aging combine with NO to form peroxynitrite, a powerful oxidant, which is increased in the arterial media of aging vessels.

NO may be obtained from endogenous and exogenous sources. For example, eNOS activity is an endogenous pathway for NO production. Exogenous sources include arginine, NONOtes, NO adducts (such as S-nitrosothiols), organic nitrates (such as nitroglycerine, [NTG]), and inorganic nitrites. However, these exogenous sources have yet to reach clinical usage. Further, there are challenges in using many exogenous NO sources. For example, organic nitrates require activation by ADH enzymes to release NO, leading to the development of tolerance and toxicity.

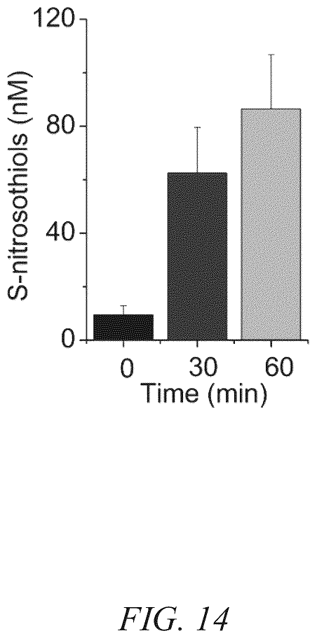

In this study, the sublingual and buccal cavity medication route of SNOAC generation and administration for acute dosing was first examined. A dose of NAC and sodium nitrite with 25 μL of water was placed under the tongue of mice or rats. Specifically, a dose of 0.1 mg of nitrite and 10 mg of NAC was placed under the tongue of the subject, blood was collected varying time points, and total S-nitrosothiols (SNO) were measured by a chemiluminescence method. As shown in FIG. 14 and FIG. 4, SNO increased with increasing the time. As shown in FIG. 14 for mice and FIG. 4 for rats, SNOAC is generated in the buccal cavity and absorbed into circulation. Further, as shown in FIG. 5, mean arterial pressure (MAP) decreases in a dose-dependent manner as SNOAC increases NO bioactivity.

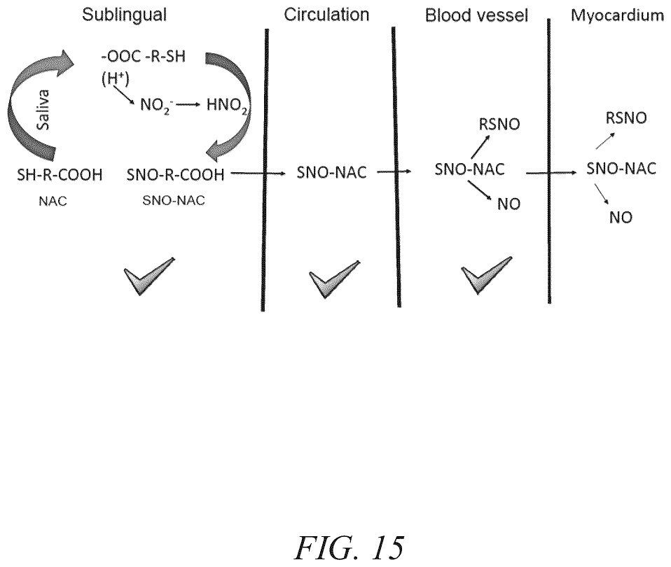

The proposed pathway of sublingual administration is shown schematically in FIG. 15. NAC dissolved in saliva decreases pH by releasing hydrogen ions. These hydrogen ions oxidize nitrite, and HNO₂ reacts with NAC products to generate SNOAC. This SNOAC then diffuses into circulation and into blood vessels, where it can cause CGMP-independent and cGMP-dependent vasodilation. Since SNOAC life span is about 30 min, it can also diffuse into cardiomyocytes where it has been shown to improve myocytes contractility and conduction.

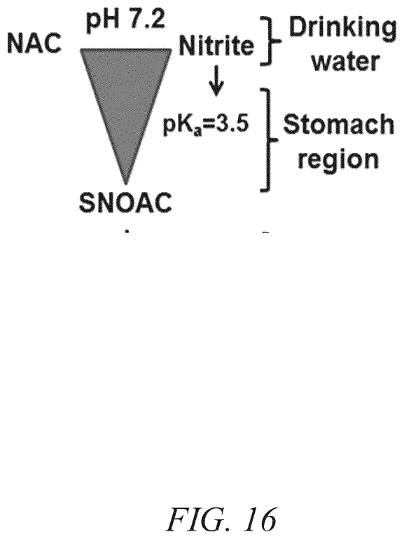

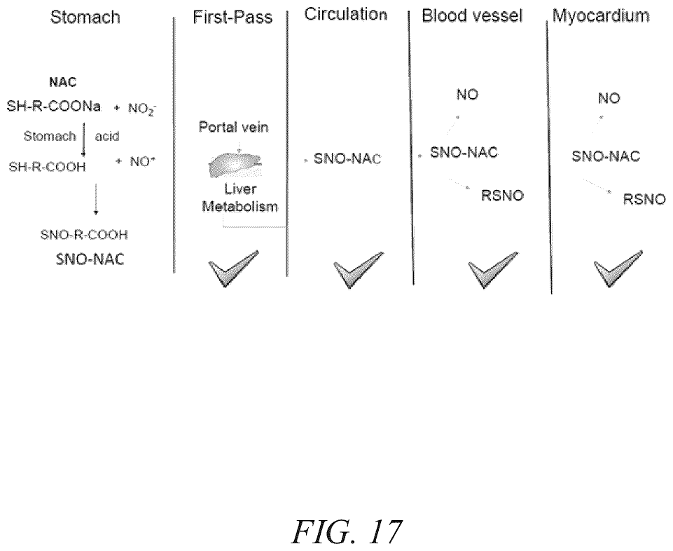

A schematic depicting the oral medication route for SNOAC generation and administration is shown in FIG. 16. In oral medication studies, NAC pH is adjusted to 7.2 and mixed with nitrite, and then administered by gavage to rats. In these studies, bioavailability was shown to be less than in sublingual administration studies. As shown in FIG. 6 and FIG. 7, first pass liver S-nitrosothiols and plasma S-nitrosothiols, and mean arterial pressure are examined, respectively. The results of oral administration of neutral NAC and nitrite are shown schematically in FIG. 17.

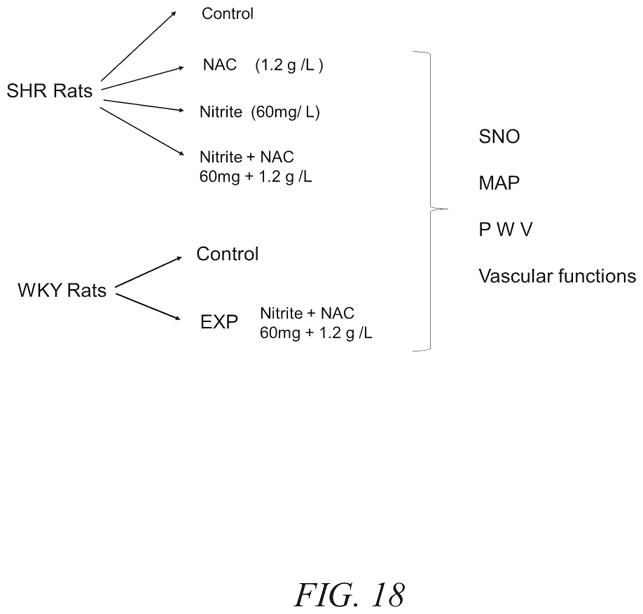

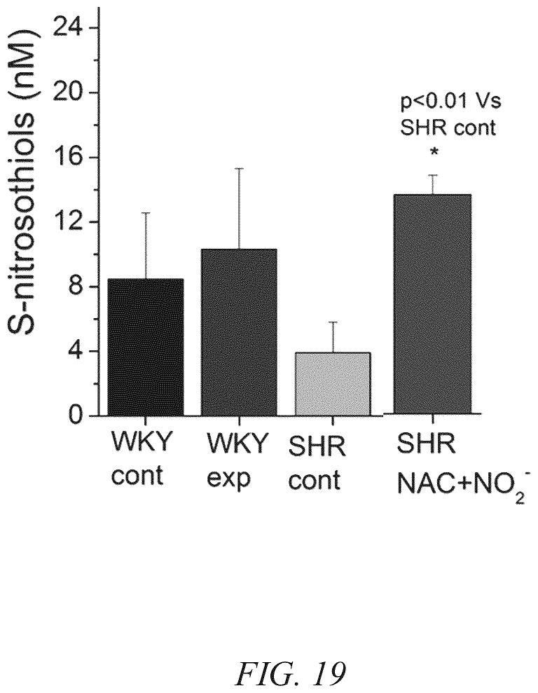

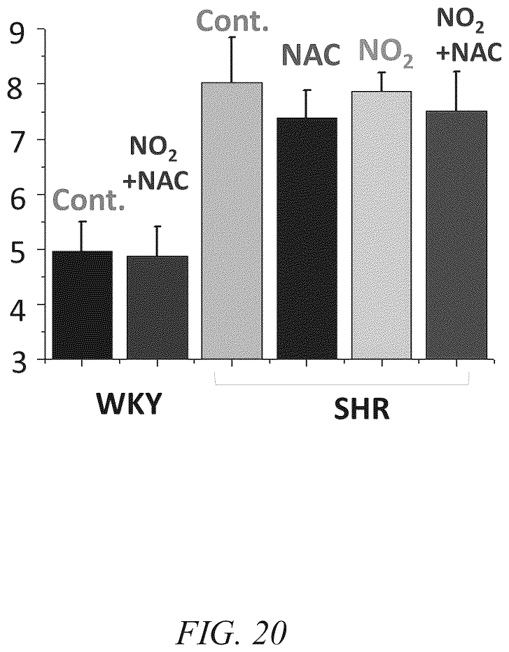

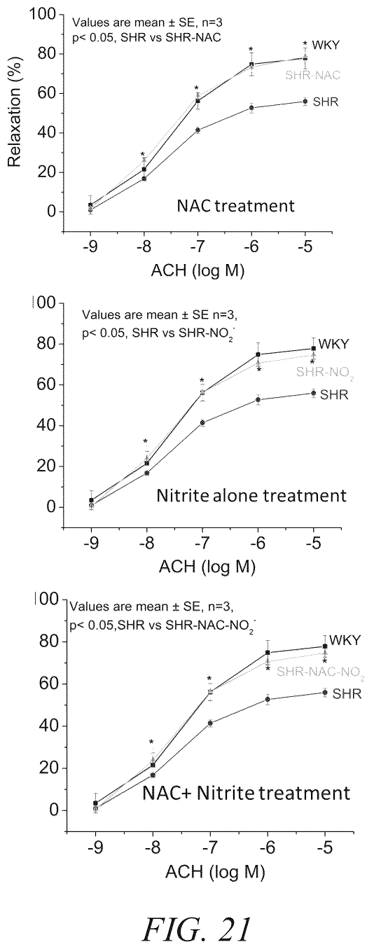

In chronic treatment studies, investigations included treatment of spontaneously hypertensive rats (SHR) and L-NAME (a nitric oxide synthase inhibitor)-induced hypertensive mice. The SHR model is useful because compounds that lower blood pressure in SHR also lower blood pressure in hypertensive humans. SHRs have been used extensively and successfully for 30 years to test medicines for their effectiveness in lowering blood pressure, and to study the mechanisms of established hypertension. The conditions of these experiments are shown in FIG. 18. The plasma S-nitrosothiol concentrations of the mice are shown in FIG. 19. Further, mean arterial pressure (FIG. 10) and arterial stiffness (FIG. 20) are examined. In FIG. 21, percent relaxation as a function of ACH are shown for each mouse group based on NAC (top panel), nitrite (middle panel), or NAC and nitrite treatment (bottom panel).

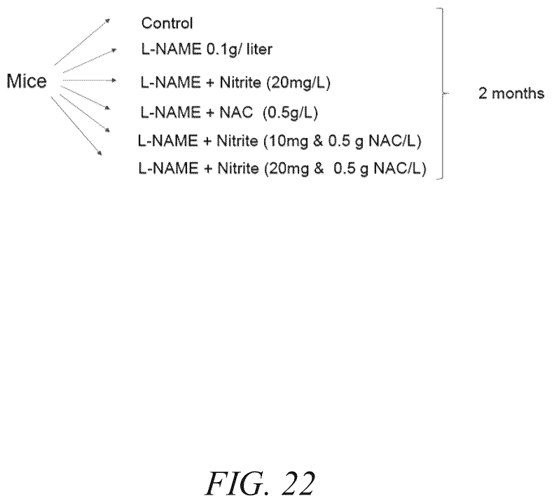

Finally, in a study with mice administered treatments according to FIG. 22 for 2 months, mean arterial blood pressure (FIG. 9) and arterial stiffness (FIG. 13) were examined.

The clinical applications include a fast releasing formula of nitrite (5 mg) with NAC (50 mg) along with stabilizing agent as a treatment for acute angina pectoris, among other conditions and diseases. The clinical applications also include a slow release formula of nitrite (5 mg) with NAC (50 mg) with additional stabilizing agents for improving overall cardiovascular function and treating angina and resistant systemic hypertension.

### Prophetic Example 7: Clinical Applications for Formulations

We will recruit participants from the UAB campus. The flyers for recruitment of participants will be posted with brief details of the study in places at elevators, breakrooms and notice boards. The participants who respond favorably will be pre-screened by phone conversation prior to in-person conversation. Patients will schedule an in-office visit over the phone for the follow-up screening process. In the follow-up screening process, the prospective participants will be invited to the study room to check their medical records as well as to measure blood pressure as one criterion for eligibility. Urine pregnancy tests will be administered during screening to women of childbearing years. The participants will have an opportunity to learn about the procedure. Consent forms will be handed to the participants who meet the criteria for signing voluntarily if they are able to, or their legally authorized representative is present. The process will take place in the same room to minimize chances of anyone overhearing the discussion.

A study will be undertaken, where the optimum levels of 2.5 mg and 5 mg sodium nitrite and fixed amount of 50 mg NAC will be used. Subjects will be divided into two groups. Group 1 will be tested with the low dose of 2.5 mg sodium nitrite and the 2nd group with a 5 mg sodium nitrite dose.

Participants will abstain from eating or drinking except water for at least 6-8 hours before the study. The participants will arrive in the morning hours to the study room. The procedure will be conducted in a quiet, air-conditioned room (21° C.). The attending physician will explain the study procedure once again, and once the participant is comfortable with the study, a peripheral intravenous needle catheter will be inserted into the antecubital fossa of the right arm to draw a blood sample. Approximately 5 mL of blood will be drawn in heparin vacutainers for the measurement of baseline plasma nitric oxide species. The FDA approved Mobile-O-graph blood measurement apparatus will be used to measure the blood pressure (BP) parameters. The cuff will be wrapped around the left arm and the measurement time will be set for 120 minutes. The blood pressure parameters will be automatically measured at 2 minutes intervals. The first 30 minutes of measurement will be considered as a baseline BP before observing the drug effect.

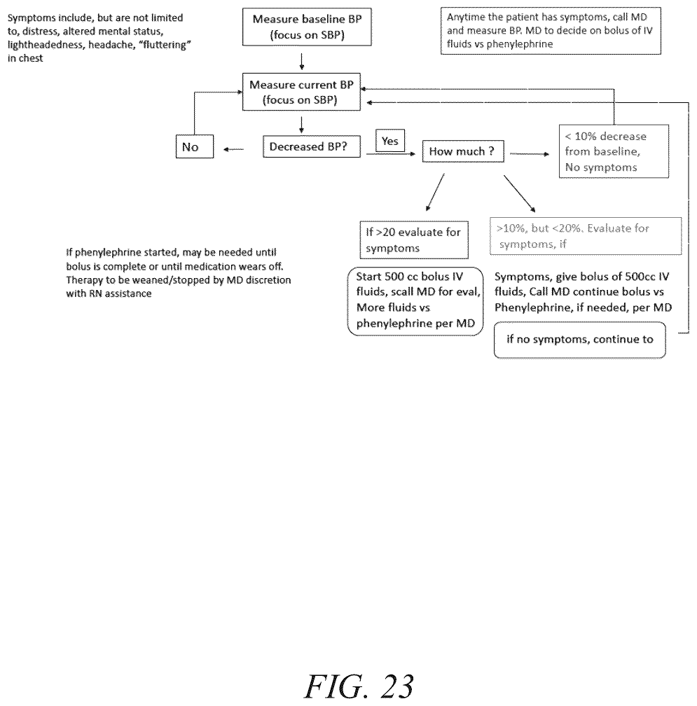

Meantime, the drug will be prepared by weighing 2.5 mg or 5 mg dry sodium nitrite powder, 50 mg dry NAC powder separately on clean butter paper using micro-balance (device that measures the sample weight in micrograms). This balance will be used exclusively for this purpose until the study is completed. These two chemicals will be mixed together just before use. Once the baseline BP measurement is completed, the drug mixture will be placed under the tongue of the subject, where saliva with the drug mixture generates SNOAC. The participant will be asked to hold the mixture against the tongue. The powder easily dissolves in saliva. The subjects will be asked not to swallow the saliva at least for 30 min, though holding the drug in the mouth will not be compulsory and the subject may swallow the drug anytime if uncomfortable. The duration the drug stays in the mouth will be recorded, and blood pressure measurements will not be interrupted while dosing. At 30 minutes after dosing, another 5 mL of blood will be drawn. The participants will be then asked to swallow the drug (if not already swallowed) that consists of unreacted nitrite, NAC and SNOAC after 30 min. These compounds are believed to be absorbed into systemic circulation from the gastrointestinal track. The major portion of SNOAC undergoes first-pass hepatic clearance before entering into circulation. Throughout the study, blood pressure monitoring will be performed according to the flowchart of FIG. 23, with appropriate actions taken if blood pressure falls below established safe levels.

The measurements will be continued until the blood pressure returns to the baseline level, generally by 90 minutes. Blood (5 mL) will be drawn again at the end of the study (15 mL total). Administration of nitrite and NAC individually at these concentrations will not be expected to change any blood NO chemistry or the systemic blood pressure. Hence, the effects of nitrite and NAC individually will not be investigated in this study. Placebo control will not be needed for this study because the baseline parameters will serve as controls. The drug effect will be assessed as the difference in pretreatment and post-treatment values. The participant will be under observation for another 1 hour in the same room.

A study investigator will be in touch with the participants for next couple of days by phone to know whether the participation in the study has caused any headaches, any irritation in the buccal cavity, bruises at the catheter injection site and any health concerns.

The blood pressure will be continuously monitored throughout the procedure. In case the BP decreases to below the safety level, the attending physician will be available to take necessary steps to mitigate this side effect by administering isotonic liquids and/or phenylephrine as shown in FIG. 23. In particular, if the blood pressure drops from the safety level, the attending physician provides a necessary medical assistance. The first line of treatment will be administrating 500 cc IV fluids through the forearm catheter while monitoring the blood pressure. The second line of treatment will be by administering a vasopressor agent, phenylephrine, at the doses of 0.1 μg to 1.5 μg/kg/min through IV fluids or direct administration through the peripheral catheter. The lifespan of the drug in the blood milieu is less than two hours following the administration. It spontaneously decays to nitric oxide and acetylcysteine disulfide. The life span of nitric oxide is less than 30 seconds. Therefore, the subject would be on fluids and/or phenylephrine for the maximum period of two hours.

Within 30 seconds of collection, the blood will be centrifuged at 5800 revolutions per minute (RPM) for 3 minutes in a small portable centrifuge. A volume of 2 mL of clear plasma, free from hemolysis, will be transferred immediately to dark colored microtubes that contain 0.1 mM diethylenetriaminepentaacetate (DTPA) and 6.25 N-ethylmaleimide. An aliquot of the sample (1 mL) will be treated with 5% 1 M HCl sulfanilamide in a ratio of 9:1 to remove nitrite for S-nitrosothiol (SNO) analysis. These samples will then be stored in liquid nitrogen until analysis. SNO in sulfanilamide-treated plasma samples will be determined by a chemiluminescence method. Plasma nitrite also will be determined by the chemiluminescence method.

### Conclusions

It is to be understood that any given elements of the disclosed embodiments of the invention may be embodied in a single structure, a single step, a single substance, or the like. Similarly, a given element of the disclosed embodiment may be embodied in multiple structures, steps, substances, or the like. The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving all such objectives. Any statements in this description criticizing the prior art are not intended to limit what is claimed to exclude any aspects of the prior art. Additionally, the disclosure shows and describes certain embodiments of the processes, machines, manufactures, compositions of matter, and other teachings disclosed, but it is to be understood that the teachings of the present disclosure are capable of use in various other combinations, modifications, and environments and is capable of changes or modifications within the scope of the teachings as expressed herein. Any section headings herein are provided only for consistency with the suggestions of 37 C.F.R. § 1.77 or otherwise to provide organizational queues. These headings shall not limit or characterize the invention(s) set forth herein.

1. A method of improvement, treatment, or prevention of a condition that is benefited by increased levels of nitric oxide, the method comprising: co-administering N-acetylcysteine (NAC) and nitrite to a subject in need thereof, and thereby producing S-nitrosoacetylcysteine (SNOAC).

2. The method of claim 1, wherein the condition is associated with low levels of nitric oxide.

3. The method of claim 1, wherein the administered SNOAC is generated when the nitrite and NAC are contacted with a bodily fluid of the subject.

4. The method of claim 1 comprising administering the nitrite and NAC sublingually, buccally, by ingestion, or by a combination of two or more of the foregoing.

5. The method of claim 1, wherein the nitrite and NAC are orally, buccally, or sublingually administered as a slow-dissolving tablet.

6. The method of claim 1, wherein the nitrite and NAC are orally, buccally, or sublingually administered as a rapid-release tablet.

7. The method of claim 1, wherein sufficient SNOAC is produced to effectively decrease blood pressure in the subject.

8. The method of claim 1, wherein the nitrite is sodium nitrite.

9. The method of claim 1, wherein the condition is ischemic heart attack, congestive heart failure, systemic hypertensive crisis, or a combination of two or more of the foregoing.

10. The method of claim 1, wherein the condition is: systemic hypertensive disease, vasoconstriction, poor circulation, erectile dysfunction, coronary artery disease, heart attack, stroke, atherosclerotic disease, vascular platelet adhesion, vascular platelet aggregation, monocyte adhesion to endothelium, leukocyte adhesion to endothelium, age-associated drop in endothelial function, peripheral artery disease, memory decline, decline in physical performance, or a combination of two or more of the foregoing.

11. The method of claim 1, wherein administration occurs on a regular schedule for at least 30 days.

12. The method of claim 1, wherein nitrite is administered at the molar equivalent of about 2.5 to about 10 mg of sodium nitrite per 75 kg of the subject; and NAC is administered at about 50 mg to about 100 mg of NAC per 75 kg of the subject.

13. The method of claim 1, wherein the administered SNOAC is generated when the nitrite and NAC are contacted with saliva of the subject.

14. The method of claim 1, wherein the administered SNOAC is generated when the nitrite and NAC are contacted with gastric acid of the subject.

15. The method of claim 1, wherein the SNOAC diffuses into circulation of the subject.

16. The method of claim 1, wherein nitrite is present at about 1 to about 10 mg in the form of sodium nitrite; and wherein NAC is present at about 50 mg to 100 mg.

17. The method of claim 16, wherein the subject is an adult human.

18. The method of claim 1, wherein the condition is congestive heart failure, and the nitrite and NAC are administered sublingually, buccally, by ingestion, or by a combination of two or more of the foregoing.

19. A method of treatment or prevention of a condition that is benefitted by increasing levels of nitric oxide, the method comprising administering S-nitrosoacetylcysteine (SNOAC) to a subject in need thereof.

20. A pharmaceutical composition for increasing levels of nitric oxide (NO) in a subject, the composition comprising: inorganic nitrite and NAC under moisture-free conditions.

21. A method of generating SNOAC, comprising: mixing nitrite and NAC in water.

22. A composition for generating SNOAC, the composition comprising nitrite and NAC under moisture-free conditions.
